(19) United States
(12) Patent Application Publication
(10) Pub. No.: US 2006/0195064 A1
(43) Pub. Date: Aug. 31, 2006

(54) Portable apparatus for peritoneal dialysis therapy

(75) Inventors: Kulwinder S. Plahey, Martinez, CA (US); Frank L. Hedmann, Volkach (DE); Stephan Klatte, Wurzburg (DE); Thomas I. Folden, Alamo, CA (US)

Correspondence Address: FISH & RICHARDSON P.C., PO BOX 1022, MINNEAPOLIS, MN, 55440-1022, US

(73) Assignee: Fresenius Medical Care Holdings, Inc., Lexington, MA (US)

(21) Appl. No.: 11/069,195

(22) Filed: Feb. 28, 2005

(51) Int. Cl. A61M 1/00

(52) U.S. Cl. 604/151

(57) ABSTRACT

A portable peritoneal dialysis apparatus having (1) a hinged door for enclosing a disposable cassette that seals tightly shut using air pressure; (2) accurate pressure sensing of pressures applied to the patient through an enclosure in the disposable cassette; (3) two pumps that can operate separately or in tandem actuated by two separate stepper motors; and (4) a touch screen user interface where indicia of the operating mode is always visible along with indicia for the other possible operating modes and the mode can be changed by touching one of these indicia.

### BACKGROUND OF THE INVENTION

The present invention relates generally to apparatus for the treatment of end stage renal disease. More specifically, the present invention relates to portable apparatus the performance of peritoneal dialysis.

Dialysis to support a patient whose renal function has decreased to the point where the kidneys no longer sufficiently function is well known. Two principal dialysis methods are utilized: hemodialysis; and peritoneal dialysis.

In hemodialysis, the patient's blood is passed through an artificial kidney dialysis machine. A membrane in the machine acts as an artificial kidney for cleansing the blood. Because the treatment is extracorporeal, it requires special machinery and a visit to a center, such as in a hospital, that performs the treatment.

To overcome this disadvantage associated with hemodialysis, peritoneal dialysis (hereafter “PD”) was developed. PD utilizes the patient's own peritoneum (a membranous lining of the abdominal body cavity) as a semi-permeable membrane. With its good perfusion, the peritoneum is capable of acting as a natural semi-permeable membrane.

PD periodically infuses sterile aqueous solution into the peritoneal cavity. This aqueous solution is called PD solution, or dialysate for short. Diffusion and osmosis exchanges take place between the solution and the blood stream across the peritoneum. These exchanges remove the waste products that the kidneys normally excrete. The waste products typically consist of solutes like urea and creatinine. The kidneys also function to maintain the proper levels of other substances, such as sodium and water, which also need to be regulated by dialysis. The diffusion of water and solutes across the peritoneal membrane during dialysis is called ultrafiltration.

In continuous ambulatory PD, a dialysis solution is introduced into the peritoneal cavity utilizing a catheter, normally placed into position by a visit to a doctor. An exchange of solutes between the dialysate and the blood is achieved by diffusion.

In many prior art PD machines, removal of fluids is achieved by providing a suitable osmotic gradient from the blood to the dialysate to permit water outflow from the blood. This allows a proper acid-base, electrolyte and fluid balance to be achieved in the body. The dialysis solution is simply drained from the body cavity through the catheter. The rate of fluid removal is dictated by height differential between patient and machine.

A preferred PD machine is one that is automated. These machines are called cyclers, designed to automatically infuse, dwell, and drain PD solution to and from the patient's peritoneal cavity. A cycler is particularly attractive to a PD patient because it can be used at night while the patient is asleep. This frees the patient from the day-to-day demands of continuous ambulatory PD during his/her waking and working hours.

The treatment typically lasts for several hours. It often begins with an initial drain cycle to empty the peritoneal cavity of spent dialysate. The sequence then proceeds through a succession of fill, dwell, and drain phases that follow one after the other. Each phase is called a cycle.

Unlike hemodialysis machines, which are operated by doctors or trained technicians, PD machines may be operated by the patient. Therefore the most commonly used touch screen user interface has to be simple and be free of many of the confusing touch screen menu trees common in prior art hemodialysis and PD machines. Furthermore, many PD patients travel, which means taking their PD apparatus with them in a car, train or plane. It is not always convenient in a hotel, for example, to have the PD equipment in a position above or below the patient. Often the best place for the equipment is on a nightstand next to the bed, which may be at approximately the same level as the patient.

Thus, it is desirable that the PD equipment be rugged, lightweight and portable, and be capable of use in many locations relative to the patient, such as at the same level as the patient as well as above or below. Also the touch screen user interface must be clear and easy to use for the patient. Moreover, the physical operation of the PD machine must not require physical strength, as PD patients are often in a weakened condition. And finally, of paramount importance is patient safety. For example, very accurate monitoring of pressure in the lines is extremely important so no harm comes to the patient.

The intent of this invention is to provide improved PD equipment with a clearer touch screen user interface, improved pressure monitoring and one that better suited for the demands of the traveling PD patient and the patient in a weakened condition.

### SUMMARY OF THE INVENTION

Briefly, the invention relates to an apparatus for pumping fluids between a peritoneal dialysis machine and a patient in order to perform peritoneal dialysis upon the patient. The apparatus includes a pair of diaphragm pumps, each having a variable stroke, adapted to be connected between the peritoneum of a patient and fluid-containing chambers.

The fluid-containing chambers include one for receiving output fluids from the patient and one containing fluids to be pumped into the patient. The apparatus further includes a stepper motor coupled to each diaphragm pump to bidirectionally actuate the pump. The stepper motors control the variable stroke of the piston of each pump so as to accurately stroke the pump in predetermined increments and at a predetermined speed to pass precise amounts of fluid between the patient and the apparatus during predetermined times. The stepper motor control is capable of operating the pair of pumps either in tandem or in opposing directions.

The apparatus of the invention further includes two substantially flat surfaces adapted to receive and hold a disposable cassette which is at least partially flexible, and which has predetermined flow paths. When placed into the machine, the cassette is aligned with the two surfaces. One of the flat surfaces is fixed and the other is hinged to the fixed surface, so that when the hinged surface is closed against the fixed surface, the cassette is held in alignment with the flat surfaces. A clamping mechanism including an inflatable pad is disposed in alignment with the two surfaces when the hinged surface is closed, for compressing together the two surfaces with the cassette in between, aligned and in tight engagement with the two surfaces. The clamping mechanism is inflated with hydraulic pressure to maintain the surfaces tightly engaged with the cassette.

The invention also includes a method of operating a peritoneal dialysis unit having a touch screen display that includes a mode-indicating portion and an operation descriptive portion. The mode-indicating portion has a plurality of touch sensitive indicia indicating the mode in which the machine is operating. The display is used to keep a patient continually informed of which of at least three operating modes the machine is operating in, the possible modes including treatment, diagnostics and data modes, as the operation descriptive portion changes to display details of a specific operation being carried out within the one mode. The indicia for each of the three operating modes is always visible to the patient while the machine is operating in the selected mode.

The operating mode is selected by the patient touching one of the touch-sensitive indicia to select a current operating mode. The indicia for that mode is highlighted in response to that mode being selected.

The operation descriptive portion of the display, describing the operation of the machine within the selected operating mode, is displayed or changed without changing either the display of the indicia for each of the three operating modes, or changing the highlighting of the selected indicia. The user changes the mode of operation of the machine by touching a different indicia, thereby highlighting the newly selected indicia and at the same time, unhighlighting the previously selected indicia for the prior mode of operation.

The apparatus of the invention further includes a removable cassette having a flexible fluid-containing enclosure which, during the operation of the machine, contains fluid. The cassette is secured in the machine by a holding mechanism and a pressure sensor is in registration and intimate contact with the fluid-containing enclosure within the cassette. Then changes in pressure within the enclosure will be sensed and measured by the pressure sensor. The pressure sensor is connected to an electronic control for the machine so that the operation of the machine can be changed in response to changes in pressure sensed by the pressure sensor.

The disposable cassette includes a flexible enclosure adapted to contain a fluid, along with ingress and egress passageways connected to the flexible enclosure to conduct fluid into and out of the enclosure to and from the patient. The flexible enclosure has a surface located on the outside of the disposable cassette, adapted to mate with a pressure sensing device to measure the pressure of the fluid contained in the enclosure.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of the PD apparatus of the invention;

FIG. 2 is a perspective view of the cassette holder of the PD apparatus of the invention;

FIGS. 3A and 3B are exploded perspective views of the cassette holder of the PD apparatus of the invention;

FIG. 4 is a front view of a cassette used in the apparatus of the invention;

FIGS. 5A-5L illustrate various fluid flow paths through the cassette used in the PD apparatus of the invention;

FIG. 6 is a schematic and block diagram of the electronic operation of the PD apparatus of the invention; and

FIGS. 7 and 8 illustrate the user interface of the invention.

Numbers referring to the same items in several drawings will bear the same reference numbers.

### DETAILED DESCRIPTION OF THE PREFERRED EMBODIMENT

### The Door Sealing Mechanism

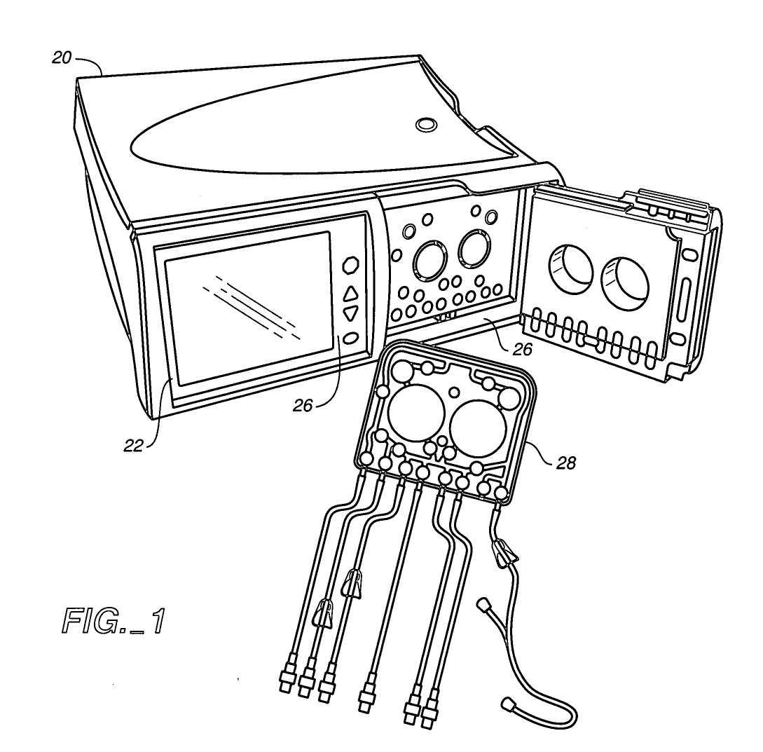

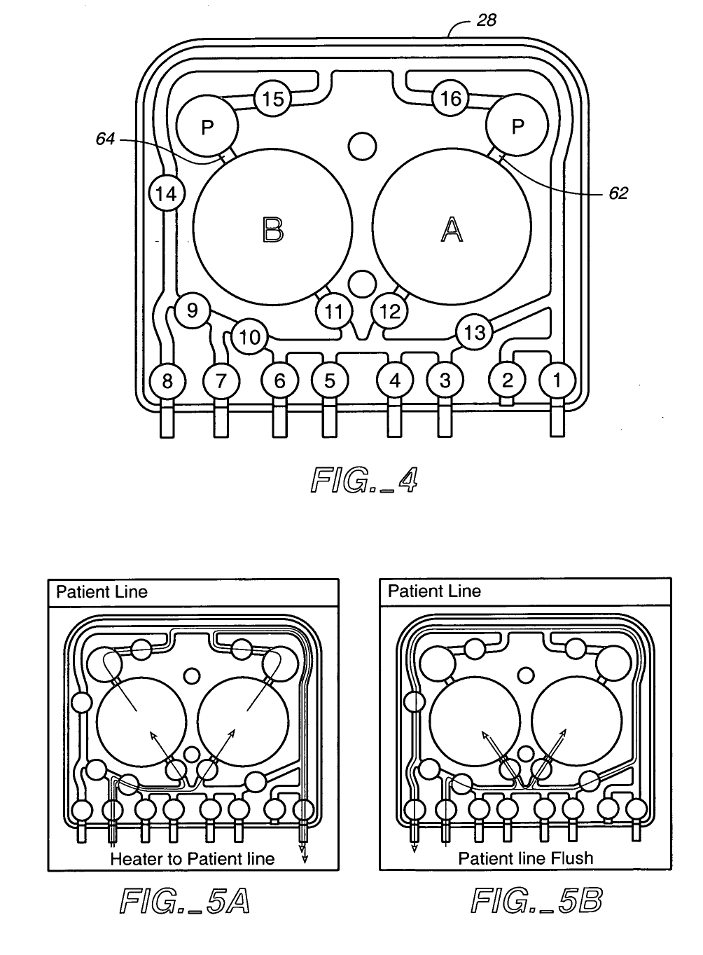

Referring to FIG. 1, the portable PD apparatus of the invention is shown. The housing 20 holds a touch screen 22, along with additional control buttons 26 operated by the patient. The cassette holder includes a hinged door 24 and a cassette support area 26. The cassette 28, shown in FIG. 4, fits into the cassette support area 26. A cassette is inserted into the support area 26 and the door 24 is closed upon the cassette and securely latched, as will be described later.

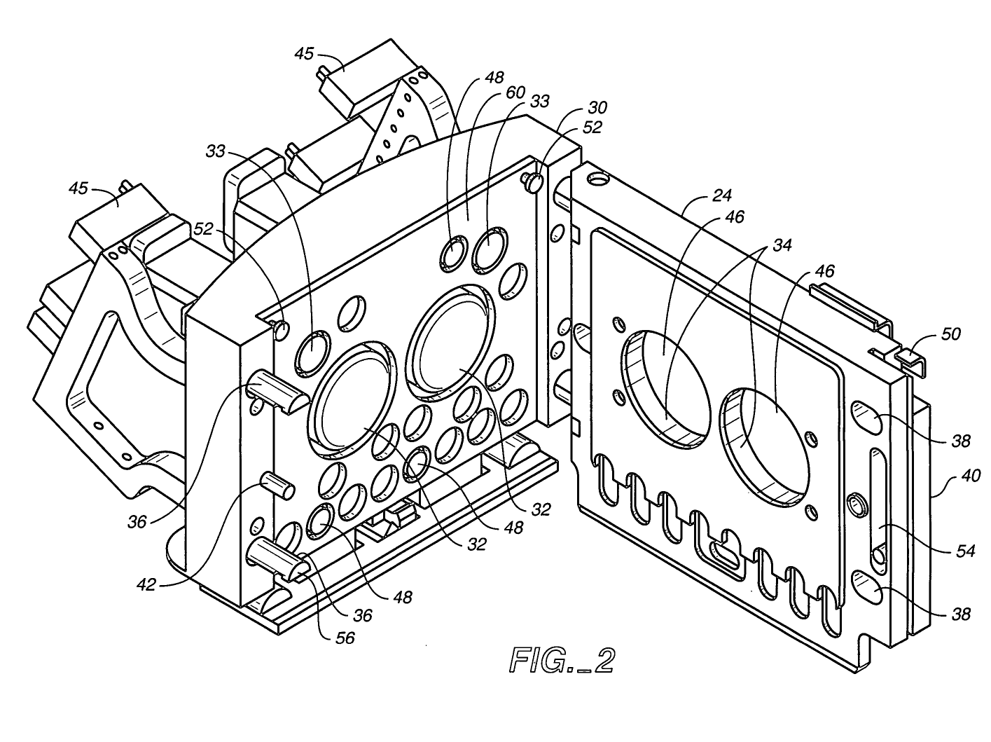

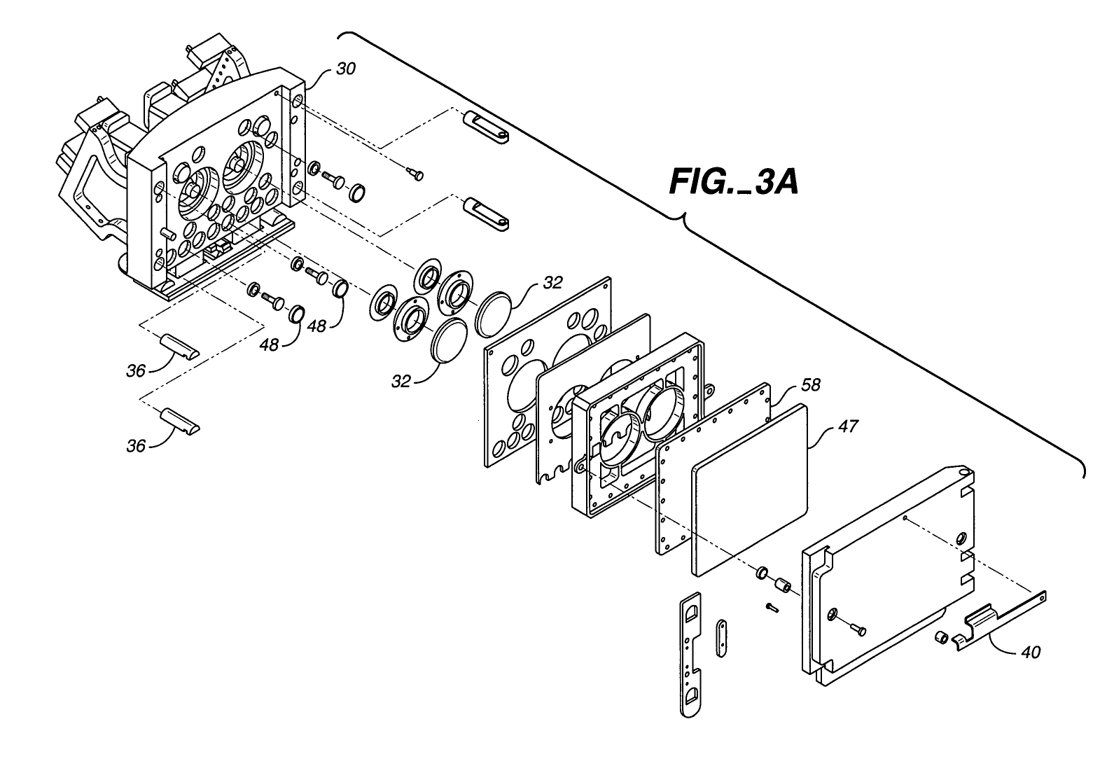

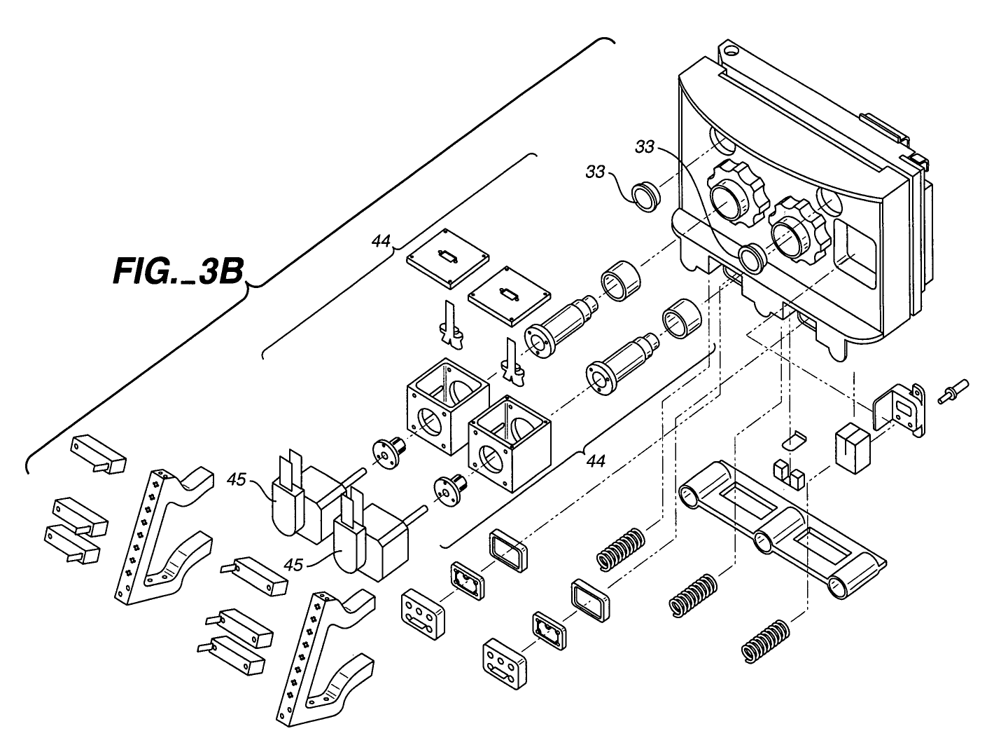

Referring to FIGS. 2, 3A and 3B, the cassette enclosure 60 will now be described in detail. Essentially, the cassette enclosure 60 consists of a base 30 and door 24 hinged to the base 30 on the right side, as shown. Base 30 incorporates two pumps 44 having exposed mushroom heads 32. Mating with these heads are two chambers 34 within door 24. The base 30 also includes a pair of door latches 36 that mate with holes 38 in door 24. The door also has a sliding latch 40. Microswitch 42 provides an electrical indication of whether the door is opened or fully closed.

It is necessary that a very tight, secure mechanical enclosure be provided with intimate contact with the cassette 28 (FIG. 4) when the machine is in operation. Prior art PD machines provided this tight enclosure by using a tight door latch that had to be almost forced closed by the patient. This created a problem for elderly or very ill patients who lacked the strength to close the door. Alternatively, in other prior art PD machines, cassettes were inserted using a complicated mechanism, similar to a VCR, making servicing more difficult. Accordingly, the PD apparatus of this invention does not require the patient to close the door with sufficient force to make all the necessary seals. Furthermore, the cassette can be set directly into enclosure 60 without use of the more complicated, VCR-like apparatus.

Door 24 is lightly latched using latch lever 40 and latch posts 36, which loosely engage with holes 38. Although the door easily “clicks” shut, the proper seals are not made by this closing. To insure that the cassette 28 is in intimate and sealed contact with both the base 30 and the door 24, the PD apparatus of the invention uses an inflatable pad 47, shown in FIG. 3A. The cassette is held in place between plate 58 and cassette enclosure 60 shown in FIGS. 3A and 2, respectively. Once the door is lightly shut and latched by the patient, and the system receives a signal to that effect from switch 42, air is pumped into pad 47, forcing the door 24 and the base 30 against the cassette (shown in FIG. 4) so that all necessary seals are correctly made. A vacuum pressure of at least about 400 lb./sq. in. is used, preferably at least 800 lb./sq. in or more can be used, but 400 lb./sq. in. is usually sufficient. This is particularly important for accurate pressure sensing, as will be described later. Yet the patient does not need to exert any force on the door or latch to close the door.

To open door 24 to load a cassette, button 50 on the top left edge of the door is depressed. This will disengage the door lock. The door then swings open from left to right. Cassette 28 (FIG. 4) may then be loaded into cassette holder by putting the top of the cassette under the locating pins 52. The bottom edge of the cassette will be snapped in. The door 24 closes from right to left pushing gently on it to automatically engage the door with latch posts 36. The catch assembly is comprised of a catch slide 40 and a catch spring (not shown). These parts are located in a machined slot 54 on the left side of the door as viewed in a closed position. As the door swings shut, the catch comes in contact with the tapered end 56 of the latch posts 36. The action of lightly pushing on the door to latch it also actuates the door safety switch 42.

Once the door safety switch is closed, the system receives an electrical signal indicating that it is ready to clamp the cassette into the cassette holder by inflating the cassette clamping inflatable pad 47 ((FIG. 3A) with approximately 37 psi pressure (which generates approximately 1000 pounds of force). This clamps the cassette 28 against the clamp pad 58 (FIG. 3A), thereby forming the correct channels within the cassette 28 for fluid control. Once the inflatable pad 47 is inflated, it pushes against both the cassette 28 and, on the other side, against plate 58. The door locking mechanism is then immobilized, preventing the door from accidentally opening or even from being opened by the patient, for safety purposes. cl The Pump

The pumps 44 (best seen in FIG. 3B) are controlled by stepper motors 45. The details of the stepper motor control will be explained later. The PD apparatus of the invention uses two modes of pumping, simultaneous and alternating. With the alternating method, while one pump is protracted, the other is retracted. Simultaneous pumping is where both pump heads extend at the same time in the same direction, and both retract at the same time.

To move fluid out of one of the chambers 34, the pump 44 mated to that chamber is moved all the way to the wall of the cassette, but not touching it. To draw fluid into one of the chambers 34, pump 44 is pulled back by one of the stepper motors 45 while building vacuum in the back of cassette 28 located within chamber 34, so as to retract the membrane of cassette 28 (not shown in FIG. 2, 3A or 3B). As the cassette membrane gets retracted, fluid is drawn into the one of the chambers A or B of the cassette 34.

For draining fluids from the patient, an alternating pumping method is employed where one pump 44 extends while the other retracts. When the pump associated with chamber A is extending, the fluid in the chamber A is pushed out into a drain line of the cassette 28. As the pump associated with chamber B retracts, fluid from the patient is drawn into chamber B. When this motion is completed, the pump associated with chamber A then retracts and draws fluid from patient while pump B protracts and transfers fluids out into the drain line. This process continues until the required volume of fluid from the patient is processed.

Initially, the pumps 44 are moved to a home position which is sensed by a conventional optical sensor, not shown. The pump controller encoder value is then set to zero. Next the pump is moved towards the cassette until it touches the cassette. This is the “OUT” position where the encoder is then set to a current encoder value less a maximum (calculated to be the maximum possible stroke, for example, an encoder count of 250). Then, the pump is moved backwards by 800 microsteps, or about an encoder count of 16000. The “HOME” position is then set to this encoder value. The stepper motor 45 next moves backward another 500 microsteps, or about an encoder count of 10,000. This is where the “IN” position is set.

Volume calculation is based on the fact that the cassette volume is a known value (based upon its physical dimensions). The volume of the pump head is also a known value (again, the calculation of this volume is based upon the physical dimensions of the pump head and chamber). If the whole mushroom head 32 is flushed against the cassette wall 46, then no fluid volume can reside in the cassette chamber. As the mushroom head 32 is moved back, however, it draws fluid into the chamber of the cassette 28 (FIG. 4). The volume of fluid drawn into the chamber is calculated by subtracting the volume of the mushroom head 32 that remains in the chamber from the volume of the chamber. To calculate how much volume of the pump head resides inside the chamber, the amount of linear travel of the pump is calculated, and this distance correlates to the distance of travel of the mushroom head. From that distance a formula is used to determine how much fluid volume still resides in the chamber.

### The Electronic Controls for the Pump

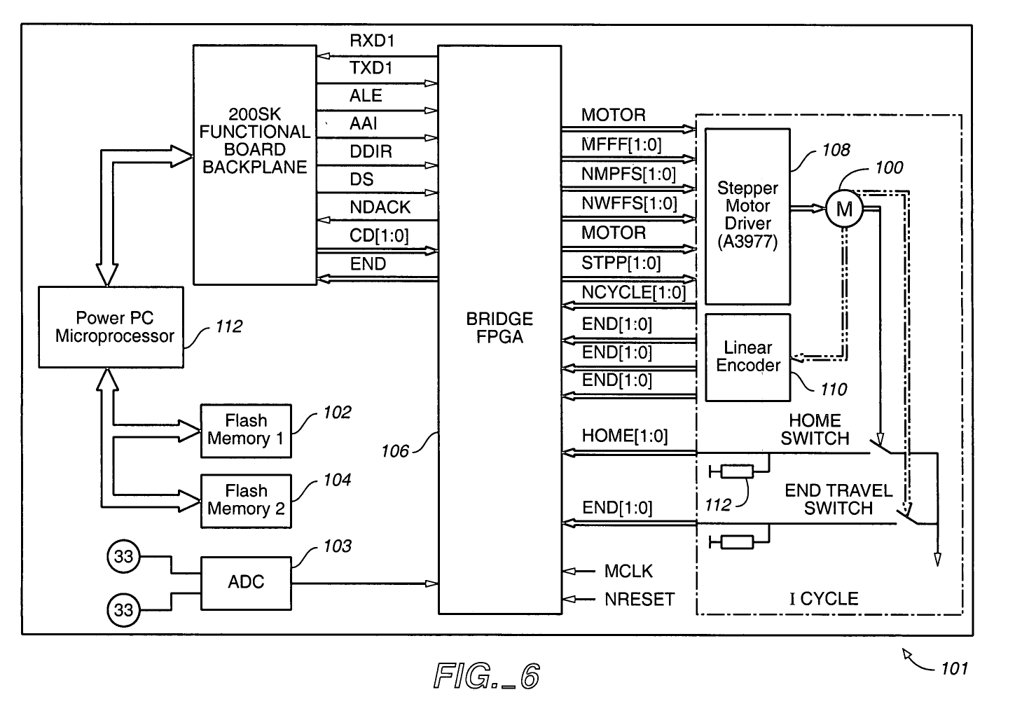

The electronics board 101 of the PD apparatus of the invention is shown in FIG. 6. Stepper motor 100, that drives each pump of the PD apparatus of the invention, are controlled conventionally using firmware with signals to stepper motor driver 108. The firmware resides in two flash memories 102 and 104. The firmware stored in flash memory 102 is used to program the bridge field-programmable gate array (FPGA) 106. The firmware stored in the flash memory 104 is used to program the MPC823 PowerPC microprocessor 112.

Referring to FIG. 2, a stepper motor 45 drives a conventional lead screw (not shown) which moves a nut (also not shown) in and out on the lead screw. The nut, in turn, is connected to a mushroom head 32 which actually makes contact with the membrane A or B on the cassette 28 (FIG. 4). The stepper motor and lead screw are chosen to provide the required force to push fluid out of the cassette following the opening of fluid paths in cassette, as will be described later. The stepper motor 45 preferably requires 200 steps to make a full rotation, and this corresponds to 0.048″ of linear travel. Additionally, an encoder measures the angular movement of the lead screw. This measurement can be used to very accurately position the mushroom head assembly.

A stepper motor controller (not shown) provides the necessary current to be driven through the windings of the stepper motor. The polarity of the current determines whether the head is moving forward or backward. Rough positioning of the piston is aided by one or more opto-sensors (not shown).

Inside the FPGA 106, there are two duplicate sets of control logic, one for each piston. The two-channel quadrature output of the linear encoder 110 (FIG. 6) is converted into an increasing or decreasing count. The overall range of this count is from 0 to ˜65,000 (or, the count can be split in half about 0, from −32,499 to +32,500). This count is required to determine the current position and subsequent movement of the piston. There is a direct correlation between actual movement of the lead screw and an encoder value.

Referring again to FIG. 6, the FPGA 106 makes a comparison between the current encoder input and a target value. This is needed for automatic movement. A single command to the FPGA 106 initiates a complete cycle that ends with the piston being moved from its current position to newly designated position. Additionally, the FPGA 106 can automatically stop the motor movement. This is desirable, for example, where the pump head reaches its end of travel (sensed by end of travel switch 112, or where the pumping action causes the pressure to be out-of-bounds. If the piston reaches an end-of-travel switch 112, the automatic movement is halted. Likewise, if a pressure sensor 48 (FIG. 2) determines that the pressure is outside of the prescribed, limited range, the motors 45 (FIG. 2) can be halted to prevent a larger excursion, which might be harmful to the patient.

Another part of the FPGA firmware allows the speed of the stepper motors 45 to be controlled, as is well known in the art. By adjusting the motor pulse duration and time between pulses, the motor can run faster or slower to get a desired speed vs. torque balance. The speed the motor runs is inversely related to the torque it is able to apply to the pump head. This adjustment allows the machine to produce the desired amount of push on the fluid in the pump chambers A or B (FIG. 4) so that it flows easily through the lines, but isn't forced so as to trigger pressure alarms or cause rupture of the lines. On the other hand, if you try to run the motor too fast, you may lose the necessary torque required on the pump head to move the fluid through the line.

In addition to the motor pulse, the FPGA 106 provides several control signals to the stepper motor controllers (not shown), for example, direction and step size. Depending on the values sent from the flash memories 102 and 104 to the FPGA 106, the step size can be adjusted between full, half, quarter and eighth steps. Also, the motor controller can be sent a continuous sequence of pulses for rapid motor movement, or just a single pulse to make a single step. This is set conventionally by registers in the FPGA 106.

### The Pneumatic System

Referring to FIG. 2,the apparatus of the invention also includes a pneumatic system, well known in the art, that provides the hydraulic pressure to operate the valves and fill pad inflatable pad 47 to seal the door closed. A compressor pump (not shown) is used to provide either air or a vacuum in corresponding reservoirs. During the pumping sequence, this air and vacuum resource is used to inflate and deflate the balloon valves 48. When inflated, a balloon valve will block the fluid from moving through the particular one of channels 1-16 (FIG. 4) of the cassette that mates with the selected one of balloon valves 48. When a balloon valve is deflated, the fluid can move freely through that particular channel controlled by that balloon valve.

### The Pressure Sensors

Referring to FIGS. 2 and 4, a very important requirement of the PD apparatus of this invention is the accurate measurement and control of pressure between the fluid reservoirs and the patient. If the pressure on a line to the patient increases above alarm limits, serious harm can be caused to the patient. The PD system itself needs to operate at pressures that far exceed the limit. These high pressures are needed for to operate the pressure sensors, balloon valves and other functions in the cassette. Therefore these pressures need to be kept independent from the pressures seen by the patient. Appropriate and reliable sealing and valving needs to be used to keep these high pressures away from the patient.

Referring to FIG. 2, to monitor the pressure in the system, two pressure sensors 33 are utilized to indirectly detect the pressure and vacuum within the patient's peritoneum. These sensors are preferably infusion pump force/pressure transducers, for example Model 1865 made by Sensym Foxboro ICT. When cassette 28 (FIG. 4) is inserted into the cassette enclosure 60, the pressure sensing areas “P” within the cassette 28 line up and are in intimate contact with the two pressure sensors 33. These sensing areas P are connected, respectively, directly to each chamber A and B through canals 62 and 64, respectively, so that when fluid moves in and out of the chambers A and B, the pressure sensors 33 can detect its presence. The cassette membrane comprising two areas marked “P” adheres to the pressure sensors 33 using vacuum pressure.

The two pressure sensors 33 are connected to a high resolution 24 bit Sigma-Delta, serial output A-D converter (ADC) 103 on I/O board 101. This ADC sends a signal from each of the two pressure sensors to the FPGA 106 on the board 101. After the data ready signal is received by the FPGA 106, the FPGA reads this ADC and transfers this data to be processed by the microprocessor 112, which in the preferred embodiment of the invention is an MPC823 PowerPC device manufactured by Motorola, Inc.

Upon completion of the flush and prime processes, as is well known in the art, the cassette will be filled with solution. At this time, the line to the patient will be completely filled with solution. The pressure at this stage is detected and will be used as base line for static pressure. At that time, the patient's head height relative to the PD machine will be determined from the differential in the pressure reading. Preferably, this pressure differential is maintained below 100 mbar.

During the drain sequence, the maximum pump hydraulic vacuum is limited to −100 mbar to prevent injury to the patient. The vacuum in the peritoneum must be held at or above this value. The position of the patient below or above the PD machine level indicated by the static pressure measurement is compensated by adjusting the level of the vacuum.

By way of example, the target vacuum of the vacuum chamber can be based on the following equation:
Pstat=static hydraulic pressure (+1 meter=+100 mbar and −1 meter=−100 mbar)

Ppatmax=−100 mbar

Pvac=target vacuum of vacuum chamber

Pvac=Ppatmax+Pstat

For example, where the patient is 1 meter above the PD machine, the differential pressure=+100 mbar; Pvac=−100 mbar+100 mbar=0 mbar.

Where the patient on same level than machine, the differential pressure=0 mbar;
Pvac=−100 mbar+0 mbar=−100 mbar.

Where the patient is 1 meter below machine, the differential pressure=−100 mbar;
Pvac=−100 mbar+−100 mbar=−200 mbar.

Since continuous flow through the various lines connected to the patient is essential to proper treatment of the patient, it is important to continuously monitor if a patient line is blocked, partially blocked or open. There are three different possible situations:

1. the patient line is open;

2. the patient line is closed; or

3. the patient line is not completely open and therefore creates an undesired flow resistance (caused, for example by the patient is lying on the line).

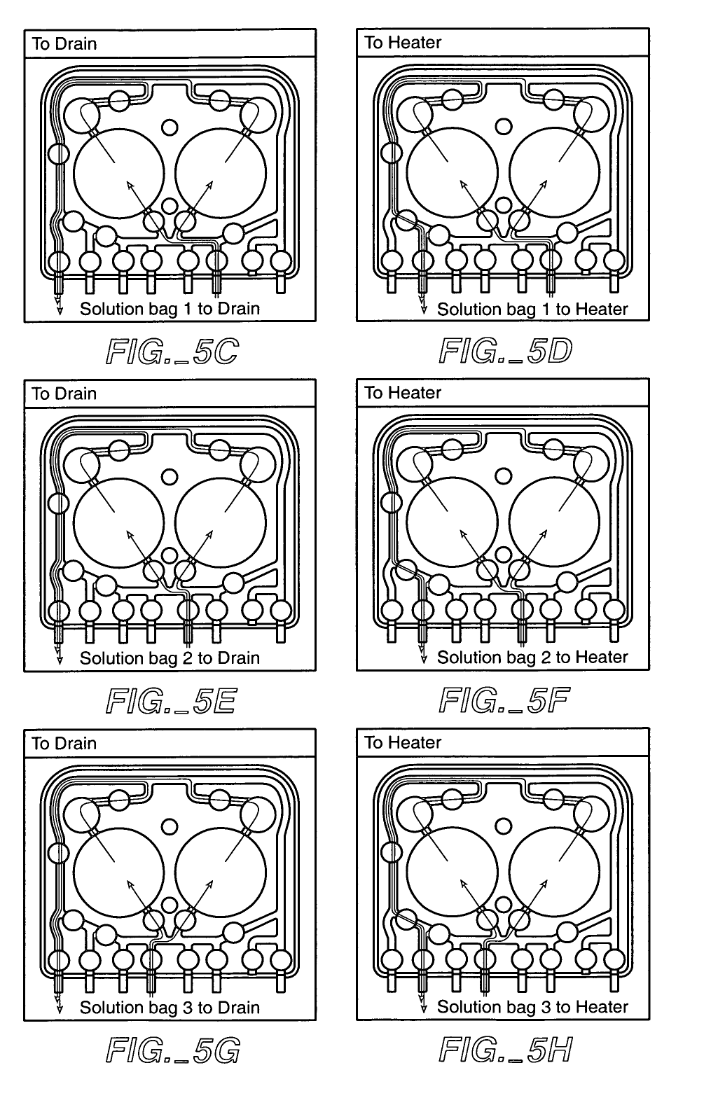

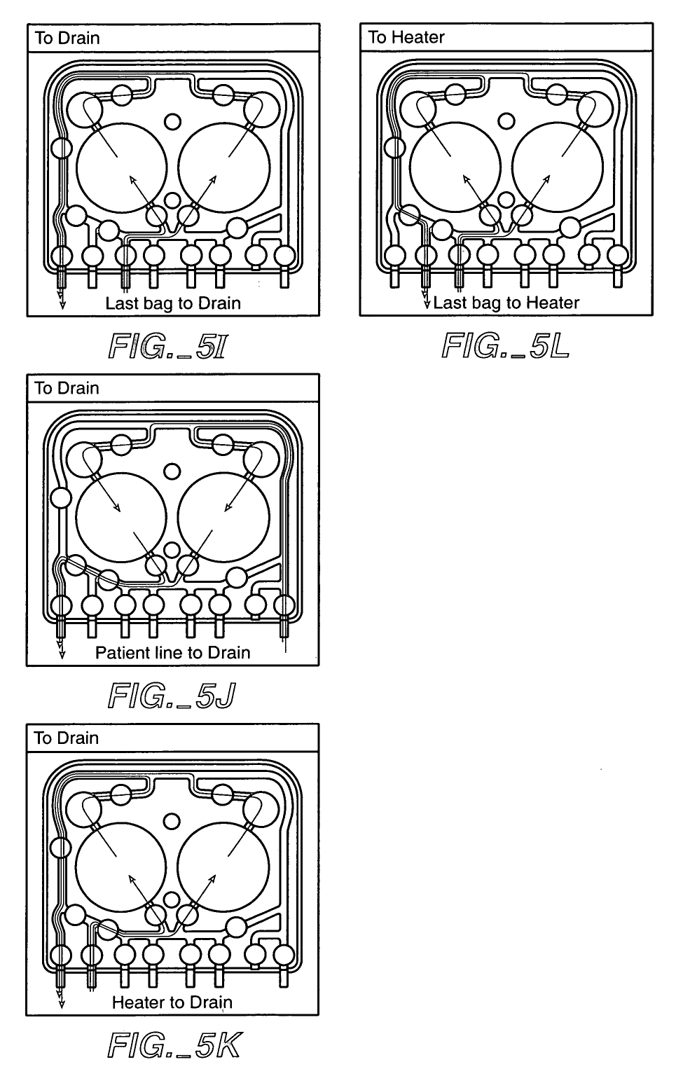

The pressure sensors 33 (FIG. 2) can be used to detect error conditions. Referring to FIG. 5A, when the pump B is protracting and thereby pumping dialysate fluid into a line that is open to patient, it is very important that the patient pressure and the encoder values are carefully monitored, using the pressure sensors 33 described above. Three possible error situations may occur, for example, as a result of the following events:

1. The patient line is open when pump B is protracting until a defined length value is reached, and the patient pressure is not increasing;

2. The patient line is closed, and the pump is not able to protract because the patient pressure increases to a defined alarm limit.

3. The pump protracts to produce an increasing patient pressure, but the pressure decreases slowly.

These error conditions may be sensed using the pressure sensors 33 of the invention, and corrective action can then be taken, either automatically or by sending an alarm to the patient, where the screen tells the patient what action to take. For example, the screen may tell the patient that he or she may be lying on a fluid line, and should move off of it.

Since the patient pressure sensors are a critical components to patient safety, it is very important to monitor whether these sensors are functioning properly. Although prior machines have attempted to accomplish this monitoring by checking the pressure readings from the sensors, such tests are not foolproof, because the varied nature of the normal, expected readings may fool one to believe that the sensors are working properly when actually they are not.

Therefore this sensor monitoring should be independent of the pressure measurements. In a preferred embodiment of the invention, the pressure sensors are monitored through an A-to-D converter (“ADC”) having two dedicated current sources, one for each sensor. Upon command, each ADC will source current (instead of acquiring data, as is usual case) and monitor how this current flows (or fails to flow) through each sensor. This independent monitoring of the pressure sensors would guarantee patient safety. Since normal treatments typically run overnight, the ability to continually double-check the very pressure sensors that monitor patient safety is indeed desirable.

### Description of Fluid Flow Through the Machine

The fluid flow through the disposable is illustrated in FIGS. 5A-5L. The PD machines of the invention utilize six fluid-processing sequences: flush, prime, drain, fill, pause and dwell. The purpose of the flush sequence is to remove air from all the lines (except the patient line) and from the cassette. This is accomplished by pumping dialysate solution through the lines to be flushed.

The prime sequence removes air from the patient line by pumping dialysate solution through the patient line. The drain sequence is used to pump dialysate solution from the patient to the drain. The fill sequence is used to pump dialysate solution from the heater bag to the patient. The pause sequence allows the patient to disconnect from the PD machine once the patient has been filled with dialysate solution. While the patient is disconnected from the machine; the machine will be transferring dialysate solution from the solution bags to the heater bag. Finally, the dwell sequence is used to allow the dialysate solution to remain for a set time in the patient. Dwell sequences are identical to pause sequences with the exception that the patient does not disconnect from the machine. While a dwell sequence is occurring, the machine will be transferring dialysate solution from the solution bags to the heater bag.

The flow sequences are shown in FIGS. 5A to 5L. Each figure contains a darker and a lighter line, each line having arrows that indicate the direction of flow. All flow diagram lines that are the same shade (darker or lighter) occur at the same time during the process.

Referring to FIG. 5A, the “Heater to Patient” line diagram, the darker lines indicate that pump A is retracting to pull dialysate solution from the heater bag. At the same time pump B is protracting to pump dialysate solution through the patient line. The lighter lines indicate that pump A is protracting to push dialysate solution to the patient. At the same time, pump B is retracting and pulling dialysate solution from the heater bag.

FIGS. 5B, 5C, 5E, 5G and 5J apply to the flush sequence as the dialysate solution comes from the supply and moves through the drain line.

FIG. 5A illustrates the prime sequence as the solution from the heater bag pushes air out of the patient, as well as the fill sequence where solution from the heater bag is pumped to the patient. FIG. 5J illustrates the drain sequence as the solution is pulled from the patient and pumped to the drain.

The pause sequence is where solution from a solution bag is pumped to the heater bag while the patient is disconnected, as shown in FIGS. 5D, 5F, 5H and 5L.

FIGS. 5D, 5F, 5H and 5L illustrate the dwell sequence where solution from a solution bag is pumped to the heater bag while the patient is still connected.

### The User Interface

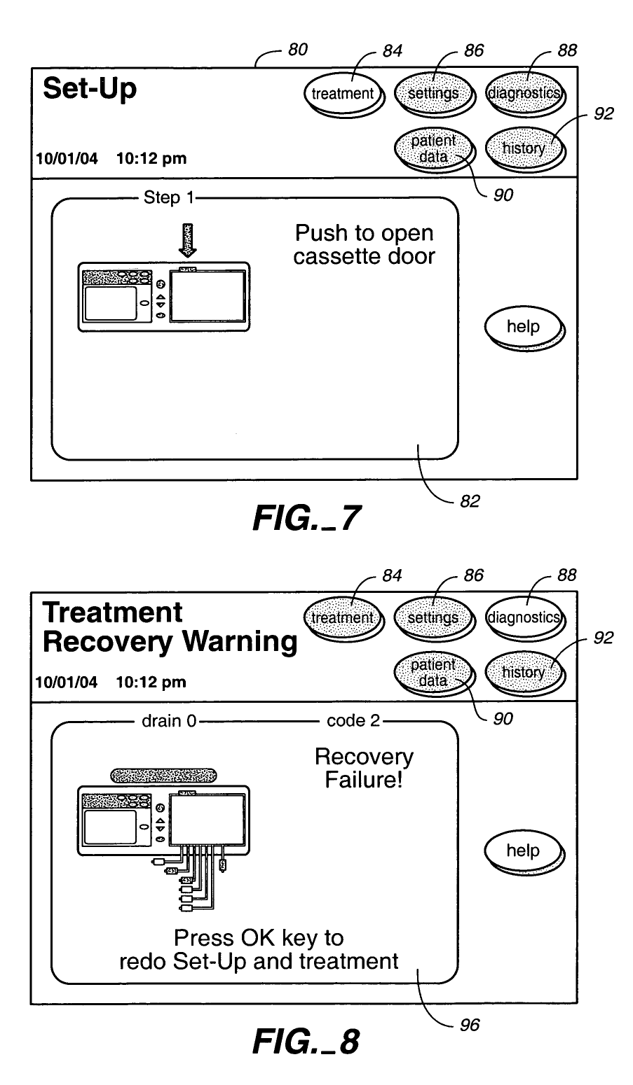

One important part of a patient-controlled PD machine is the user interface, shown in FIG. 7. A common problem with prior art machines is that the patient loses track of the mode in which the machine is operating. In the invention, the touch screen display has at least two portions: one is a mode-indicating portion 80, and the other is an operation descriptive portion 82.

The mode-indicating portion 80 has a plurality of touch sensitive indicia 84, 86, 88, 90, and 92, each indicating the mode in which the machine is operating to keep the patient continually informed of which one of at least three operating modes the machine is operating in. These modes as illustrated in the preferred embodiment shown in FIG. 7. By way of example and not of limitation, the modes may include: a treatment mode 84, during which dialysis is taking place; a settings mode 86, where the treatment type settings of the PD machine are displayed and can be modified by the patient; a diagnostic mode 88 where the operation of the machine is being diagnosed; a patient data mode 90, where patient data is displayed; and treatment history mode 92, where prior treatment of the patient is displayed.

During operation under any of these modes, the operation descriptive portion 82 of the display changes to display details of the specific operation being carried out within the selected mode. Generally, the descriptive portion shows helpful information to guide the user in operating the machine. For example, during treatment, when the treatment mode indicator is highlighted, as shown in FIG. 7, the descriptive portion 82 shows the patient that the next required step is to “Push open cassette door.” Alternatively, the descriptive portion may show the direction of fluid flow, or provide an indication of the extent of treatment completion or other description of the current stage of treatment. The same kind of descriptions are provided for various diagnostic operations which take place in the diagnostic mode.

All five illustrated mode indicia in the mode portion 80 of the screen, for each of the five operating modes of the preferred embodiment, always remain visible to the patient, with the mode that the machine is currently operating in being highlighted in some manner, as shown in FIG. 7 for the treatment mode indicator 84.

The operating mode is changed by the patient by touching one of the indicia on the screen different from the one (“treatment” in FIG. 7) that is currently highlighted. Unless there is some reason, such as safety or otherwise, that the mode must not be changed at that time, the mode will change to the new mode when the patient touches the different icon, and the newly selected icon 88, “diagnostics” as shown in FIG. 8, will be highlighted and the “treatment” icon 84 for the prior operating mode will no longer be highlighted, as shown in FIG. 8.

Then the descriptive portion 96 of the touch screen, shown in FIG. 8, will display information pertaining to the new “diagnostics” mode of operation, such as a “treatment recovery warning” shown in FIG. 8. Icons 84, 86, 90 and 92 for all the other four possible modes in the preferred embodiment will remain displayed, but not highlighted, so the patient always knows (1) what mode the machine is operating in; and (2) what other possible operating modes exist.

The invention has been described in terms of particular embodiments. Other embodiments are within the scope of the following claims. For example, steps of the invention can be performed in a different order and still achieve desirable results.

What is claimed is:

1. An apparatus for pumping fluids between a peritoneal dialysis machine and a patient in order to perform peritoneal dialysis upon the patient, comprising:
a pair of diaphragm pumps, each having a variable stroke, the pumps being adapted to be connected between the peritoneum of a patient and fluid-containing chambers;
the fluid-containing chambers including one for receiving output fluids from the patient and one containing fluids to be pumped into the patient;
a stepper motor coupled to each diaphragm pump to bidirectionally actuate the pump; and
a digital stepper motor control for controlling the variable stroke of the piston of each pump so as to accurately stroke the pump in predetermined increments and at a predetermined speed to pass precise amounts of fluid between the patient and the apparatus during predetermined times, the control being capable of operating the pair of pumps either in tandem or in opposing directions.

2. An apparatus for pumping fluids between a peritoneal dialysis machine and a patient in order to perform peritoneal dialysis upon the patient, the apparatus including a cassette enclosure comprising:
two substantially flat surfaces adapted to receive and hold a cassette which is at least partially flexible and which has predetermined flow paths, the cassette being aligned with the two surfaces, one of the substantially flat surfaces being fixed and the other being hinged to the fixed surface to hold the cassette in alignment with the flat surfaces when the hinged surface is closed; and
a clamping mechanism including an inflatable pad disposed in alignment with the two surfaces, for compressing together the two surfaces with the cassette therebetween aligned and in secure engagement when the hinged surface is closed, the clamping mechanism being inflated with hydraulic pressure to maintain the surfaces tightly engaged with the cassette.

3. The apparatus of claim 2 where in the clamping mechanism resides within the hinged surface.

4. A method of operating a peritoneal dialysis unit having a touch screen display that includes a mode-indicating portion and an operation-descriptive portion, the mode-indicating portion having a plurality of touch sensitive indicia each indicating one of the modes in which the machine can operate, the display being used to keep a patient continually informed of which one mode of at least three operating modes the machine is operating in, including treatment, diagnostics and data modes, as the operation descriptive portion changes to display details of a specific operation being carried out within the one mode, the indicia for each of the three operating modes always being visible to the patient while the machine is operating, the operating mode being selected by the patient touching one of the indicia on the screen, comprising:
touching one of the indicia to select a current operating mode in which the machine is operating, the indicia for the selected mode being highlighted in response to that mode being selected;
displaying in the operation descriptive portion of the display information concerning the operation of the machine within the selected operating mode without changing either the display of the indicia for each of the three operating modes or changing the highlighting of the indicia for the selected mode;
changing the mode of operation of the machine by selecting a different indicia, thereby highlighting a different indicia and at the same time, unhighlighting the previously selected indicia for the prior mode of operation.

5. Apparatus for measuring pressure in a peritoneal dialysis machine comprising:
a removable cassette having a flexible fluid-containing enclosure in which, during the operation of the machine, fluid is contained;
a holding mechanism for securing the cassette in a predetermined location within the machine;
a pressure sensor in registration and intimate contact with the fluid-containing enclosure when the cassette is retained in the machine, so that any changes in pressure within the enclosure will be sensed by the pressure sensor;
a connection between the pressure sensor and an electronic control for the machine so that the operation of the machine can be changed in response to changes in pressure sensed by the pressure sensor.

6. A disposable cassette for transporting fluids to and from a patient undergoing peritoneal dialysis comprising:
a flexible enclosure adapted to contain a fluid;
ingress and egress passageways connected to the flexible enclosure to conduct fluid into and out of the enclosure to and from the patient;
the flexible enclosure having a surface located on the outside of the disposable cassette, the surface adapted to mate with a pressure sensing device so as to measure the pressure of the fluid contained in the enclosure.

7. The cassette of claim 6 wherein the surface of the enclosure is circular.
